(19) United States
(12) Patent Application Publication
(10) Pub. No.: US 2008/0108860 A1
(43) Pub. Date: May 8, 2008

(54) Methods and Apparatus for Magnetic Manipulation or Retrieval

(76) Inventors: Stephen G. Bell, Rome (IT); Wayne A. Noda, Mission Viejo, CA (US); Elbert Y. Tzeng, Irvine, CA (US)

Correspondence Address: SCOTT EVANS, 1252 COUNTRY HILLS DR., SANTA ANA, CA, 92705, US

(21) Appl. No.: 11/924,139

(22) Filed: Oct. 25, 2007

Related U.S. Application Data
(60) Provisional application No. 60/864,140, filed on Nov. 2, 2006.

(51) Int. Cl. A61N 2/10; A61B 17/10; A61M 25/09; A61M 29/02

(52) U.S. Cl. 600/12; 604/164.13; 604/96.01; 606/139

(57) ABSTRACT

Apparatus and methods for minimally invasive stabilization, manipulation and/or removal of body cavities or organs in the body through the skin or natural body orifices. A magnet is delivered into an organ, the position of the organ is stabilized with an opposing magnetic retrieval device positioned outside the organ, and the organ is ligated and removed from the body through the skin or a natural body orifice. This magnetic linkage between an internal body cavity with a retrieval device may be used to hold the position of the body cavity steady during resection and aid in the removal of that structure from the body. The magnet may be a balloon filled with a magnetic gel or fluid.

### CLAIM OF PRIORITY

This application claims priority under 35 U.S.C. § 119(e) to U.S. Provisional Application No. 60/864,140, filed Nov. 2, 2006, the entire contents of which are hereby expressly incorporated by reference.

### FIELD OF THE INVENTION

The present invention relates to devices and methods for minimally invasive stabilization, manipulation and/or removal of body cavities or organs in the body through natural body orifices. More specifically, the invention relates to delivering a magnet or magnetic fluid into the body cavity or organ, fixating the position of the body cavity or organ with an opposing magnetic retrieval device, ligating and then subsequently removing the body cavity or organ through a natural body orifice.

### DESCRIPTION OF THE RELATED ART

Laparoscopic and endoscopic surgical procedures are minimally invasive procedures in which operations are carried out within the body by means of elongated instruments inserted through small entrance openings in the body. The initial opening in the body tissue, to allow passage of endoscopic instruments to the interior of the body may be a natural passageway of the body such as the esophagus or rectum. The initial opening in the body tissue to allow passage of laparoscopic instruments may be created by a tissue piercing instrument such as a trocar, a scalpel or a needle.

Laparoscopy was initially used by gynecologists to diagnose and treat conditions relating to the female reproductive organs: uterus, fallopian tubes, and ovaries. It is now used for a wider range of procedures, including operations that in the past required open surgery, and is now the preferred method in procedures such as removal of the appendix (appendectomy) and gallbladder removal (cholecystectomy). Laparoscopy allows multiple surgical instruments and tools to be inserted through separate incisions to perform procedures. Often one instrument can be used to isolate or grasp a tissue while another is used for resection or removal. However, these additional benefits are achieved at the cost of increased invasiveness. Access paths must be created for the instruments with the use of trocars requiring general anesthesia, risk of complications and infection, and increased overall recovery time for the access paths to heal. In addition, access may be difficult or contraindicated in some patients, particularly in the morbidly obese. Additionally, the abdomen must be insufflated with a gas to separate organs and the abdominal walls in order to create an operating space for the laparoscopic instruments to maneuver in. This gas insufflation can be difficult because the gas is constantly leaking and can cause additional discomfort for the patient.

Endoscopy is a form of minimally invasive procedure wherein the interior of the body is accessed and visualized through a body's natural body orifice (NO), such as the esophagus or rectum. Such access allows a surgeon or physician to view and/or treat internal portions of the orifice or internal tissues or organs which are accessible through the orifice. These procedures may be for diagnostic purposes, such as visual inspection or the removal of a tissue sample for biopsy, or the procedure may be used for treatment purposes, such as the removal of a polyp or tumor or the restructuring of tissue. While these procedures can be done using regular open surgery and/or laparoscopy, endoscopy usually may involve less pain, less risk, less scarring, and faster recovery for the patient.

However the use of endoscopy through the mouth, rectum, bladder or vagina to remove, for example, the appendix (appendectomy) or gallbladder (cholecystectomy) is not common because these types of pedunculated structures such as the appendix and gall bladder are not easily accessible from inside natural body orifices. Because surgeries through NO require compact, flexible instruments with limited mobility and degrees of operating freedom and because there may be only a single instrument operating in one or two axes, the isolation, fixation, manipulation and subsequent removal of these body organs can be difficult. It may be helpful to have other instruments that can attach to these organs and assist in organ removal. These difficulties can be compounded if little or no insufflation is used, which may further decrease maneuverability and visibility.

Therefore a device and method to locate, isolate and retract a targeted tissue in a relatively stable fixed position prior to further intervention may be helpful. Additionally the ability to manipulate an organ using separate tools other than the NO instrument to expose an optimal surgical site may facilitate resection of that organ. Even though access to these organs through natural body orifices is less traumatic and potentially safer, the devices and methods to perform these surgeries have not been available.

Thus, it would be desired to provide an improved method, with devices and systems to perform minimally invasive surgery, particularly appendectomies and cholecystectomies, through natural body orifices. Particularly, methods, devices and systems which would provide the benefits of endoscopy, such as lower invasiveness and access to deeply internal locations, with the benefits of laparoscopy.

### BRIEF DESCRIPTION OF THE INVENTION

The preferred methods, devices and systems described herein provide for minimally invasive stabilization, manipulation and removal of body cavities, organs, tissue, growths or structures in the body through natural body orifices. Accordingly, an objective of this invention is to facilitate access, retraction, stabilization, and manipulation of body cavities using magnetic retrieval devices and methods Preferably, one aspect of the invention relates particularly to accessing the appendix or gall bladder and introducing a magnet into these structures. This magnet may be magnetically coupled to a retrieval device introduced to the abdominal space through a NO, across the skin or through the skin. This magnetic linkage between an internal body cavity with a retrieval device may be used to hold the position of the body cavity steady during resection and aid in the removal of that structure from the body.

One aspect of the invention is directed to a device for stabilizing and resecting a body cavity or organ that, as embodied and broadly described herein, include a magnet configured for delivery to the inside of the body cavity, a delivery device to deliver the magnet to the body cavity, and a magnetic retriever configured to magnetically couple with the magnet through a wall of the body cavity. This coupled magnetic retriever and magnet may be used to stabilize the position of the body cavity and to control the position of the body cavity relative to the outside of the body. The body cavity may be further resected whereupon the magnetic retriever may be withdrawn from the body along with the resected body cavity.

In another aspect of the invention the magnet may be an inflatable balloon. The balloon may be initially deflated and located near the end of an inner lumen of a delivery device. The balloon may be inflated with a magnetic gel that expands the balloon outside the inner lumen of the delivery device. The delivery device may utilize a closure element to seal the balloon and detach the balloon from the delivery catheter.

In another aspect of the invention the magnetic retriever may be an elongated tubular body that has an expandable sheath attached to a distal end. The retriever may also have a retractable shaft positioned longitudinally inside the body and sheath with a magnet coupler connected to the distal end of the shaft. This magnet coupler may be designed to magnetically couple with a magnet separately positioned in the organ. The shaft is also designed to retract inside the sheath and tubular body once the magnetic coupler is coupled to the magnet. This may be helpful in drawing the magnet and the body cavity or organ into the expandable sheath.

One aspect of the invention is directed to a method of stabilizing or manipulating a body cavity including the steps of positioning a delivery device inside the body cavity, delivering a magnet to the cavity, magnetically coupling to the magnet with a magnetic retriever positioned outside the body cavity and then moving, repositioning or stabilizing the body cavity by manipulating the magnetic retriever that is coupled to the body cavity because of the magnetic attraction to the magnet. Another embodiment of this method may include filling an inflatable balloon with a magnetic gel or fluid, sealing the neck of the balloon and then releasing the balloon into the body cavity.

Additionally another aspect of the invention may be a method of resecting a body cavity or organ including the steps of positioning a delivery device, which has a magnet, inside the body cavity or organ and delivering the magnet to the cavity. Further steps may include positioning a magnetic retriever outside the body cavity or organ where the retriever has an elongated tubular body with an expandable sheath attached to the distal end. The retriever may also have a retractable shaft positioned longitudinally inside the body and sheath with a magnet coupler connected to the distal end of the shaft. This magnet coupler may be designed to magnetically couple with the magnet. The shaft may also be designed to retract inside the sheath and tubular body after the body cavity or organ has been resected. This may be helpful in drawing the magnet and the body cavity or organ into the expandable sheath so that the organ can be removed from the body.

Finally another aspect of the invention may be a system for removing body tissue from the body. This system may include a magnet designed for delivery inside the body tissue and a delivery device to deliver the magnet into the body tissue. Also included in the system is a magnetic retriever designed to magnetically couple with the magnet across a wall of the tissue. The system may include a resection device to separate the tissue from the body and a ligation device to seal the opening where the organ was removed.

For illustration, natural body orifice removal of an appendix may be described in this specification. However the devices and methods described in this specification could be similarly utilized for the removal of the gall bladder, removal of cysts, tumors, growths, fistulas, vessels, ducts, other organs using similar devices and methods through the rectum, bladder, vagina or uterus, stomach or the gastrointestinal tract. Likewise the devices and methods described in this specification could conceivably be utilized in percutaneous procedures through the skin to access the abdominal cavity to treat the structures indicated.

Certain objects and advantages of the invention are described herein. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

All of these embodiments are intended to be within the scope of the present invention herein disclosed. However, despite the foregoing discussion of certain embodiments, only the appended claims (and not the present summary) are intended to define the invention. The summarized embodiment, and other embodiments of the present invention, will become readily apparent to those skilled in the art from the following detailed description of the preferred embodiments having reference to the attached figures, the invention not being limited to any particular preferred embodiment(s) disclosed.

### BRIEF DESCRIPTION OF THE DRAWINGS

FIG. 1A is a section view of a magnet and magnet delivery device in a body cavity.

FIG. 1B is a section view of the embodiment of FIG. 1A also showing a magnet retriever.

FIG. 1C is a section view of the magnet and magnetic retriever magnetically coupled across a body cavity wall.

FIG. 1D is a section view the decoupling of the magnet delivery device from the magnet.

FIG. 2 is an illustration of a magnet positioned in an appendix.

FIG. 3 is a plan view of an embodiment of a magnetic retriever.

FIG. 4A is a section view of the magnet and magnetic retriever magnetically coupled across a wall of an appendix.

FIG. 4B is a section view of the magnetic retriever with a sheath extended over a portion of the appendix.

FIG. 5 is a side view of a magnetic gel delivery device.

FIG. 6A is a section view of the device of FIG. 6A with the balloon initially deployed.

FIG. 6B is a section view of the device of FIG. 6A with the balloon fully inflated but still coupled to the delivery device.

FIG. 6C is a section view of the device of FIG. 6A with the balloon fully deployed and decoupled from the delivery device.

FIG. 7 is a section view of a magnetic gel balloon delivered to a body cavity using a percutaneous insertion.

FIG. 8 is an illustrative view of a magnetic gel balloon delivered to a body cavity using a percutaneous insertion.

FIG. 9 is an illustrative view of a magnetic gel balloon delivered to a body cavity using endoluminal insertion.

### DETAILED DESCRIPTION OF THE INVENTION

Apparatus and methods for minimally invasive stabilization and removal of body cavities, organs, tissue, growths or structures in the body through natural body orifices are described. The use of magnetic retrieval devices and methods to facilitate access, retraction, stabilization, and manipulation of body cavities is described. Preferably, one aspect of the invention relates particularly to accessing the appendix or gall bladder and introducing a magnet into these structures. This magnet may be magnetically coupled to a retrieval device introduced into the abdominal space through a NO or through the skin. This magnetic linkage between an internal body cavity with a retrieval device may be used to hold the position of the body cavity steady during resection and aid in the removal of that structure from the body.

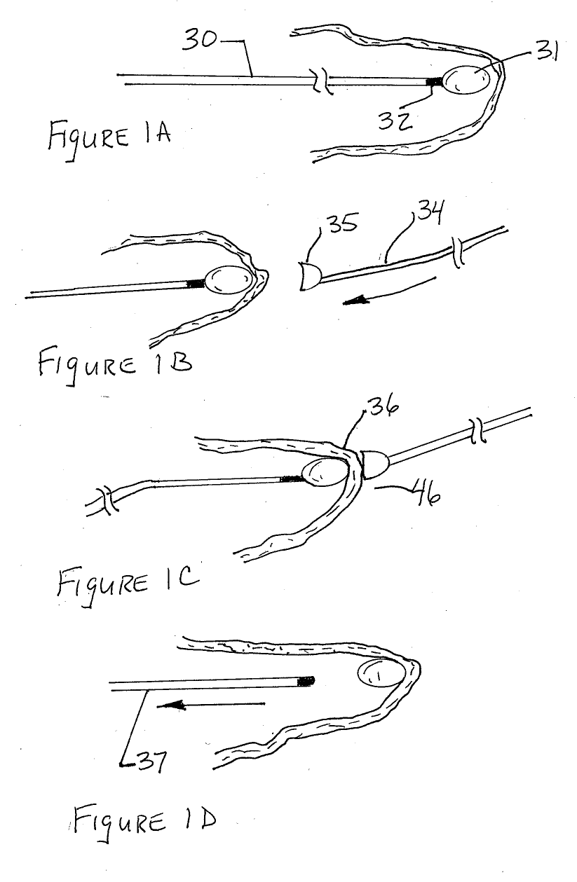

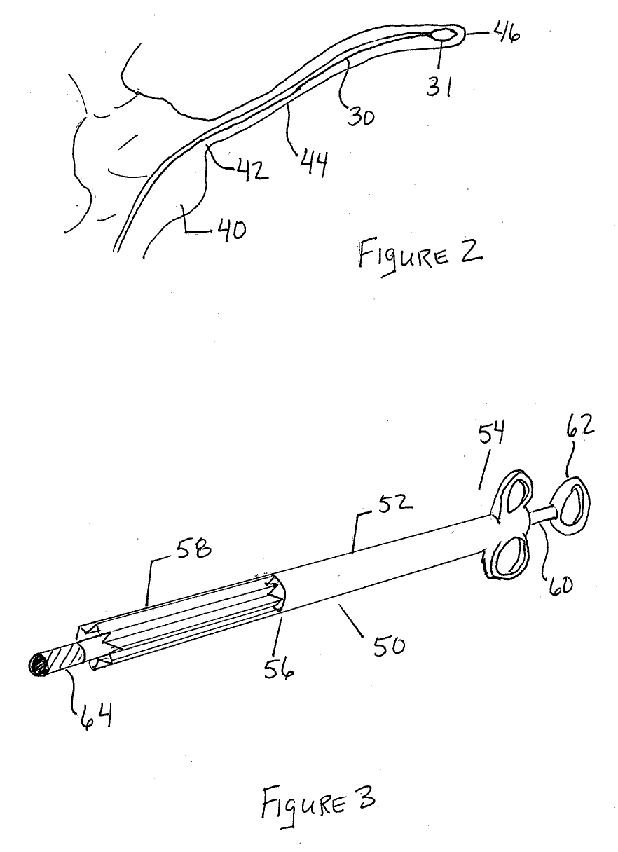

An embodiment of the current invention is shown in FIGS. 1A-D. A delivery device 30 is shown having a magnetic element 31 coupled to the tip at a union 32 as shown in FIG. 1A. This delivery device is flexible and suitable for delivery to the intended target preferably through a natural body orifice. The delivery device 30 may be a guidewire, catheter, sheath, or a trocar and may be steered to the site with fluoroscopy or using endoscopic guidance. In the case of treatment of the appendix, as shown in FIG. 2, the delivery device is designed to be introduced in the rectum and guided under fluoroscopic guidance into the cecum 40 and finally into the ostium 42 of the appendix 44. The delivery device 30 may be further placed into the tip 46 of the appendix 44. As shown in FIG. 1B, a magnetic retriever 34 having a magnetic end portion 35 with an opposite polarity to the magnetic element 31 may be inserted through a natural body orifice or through the skin of the abdomen and advanced to the wall 36 of the appendix tip 46 under fluoroscopy. The magnetic element 31 may be any ferromagnetic substance that is capable of either attracting a magnetic charge from another magnet having opposite polarity or attracting a metallic ferrous metal. The magnetic attraction of the two magnets or elements may aid in the locating and mating of the delivery device 30 and the magnetic retriever 34 through the appendix wall 36 to form a virtual continuous linkage from inside the appendix 44 to the outside of the appendix. Alternately only one of the two mating surfaces may be magnetic with the other mating surface being a ferrous metal. The magnetic attraction between the metal and the magnet may similarly aid in the locating and mating of the delivery device 30 and the magnetic retriever. As shown in FIG. 1D, the delivery device 30 may have a body 37 that may be disconnected from the magnetic element 31 and removed. The union 32 may be a screw thread, bayonet type clamp, another magnet or any other union of the two that securely joins the magnet and delivery device together yet provides for detachment capability. In one embodiment, the delivery device body 37 and the magnetic element 31 may be threaded together and a rotation of one relative to the other may facilitate their separation. Removal of the delivery device body 37 facilitates the ligation and resection of the appendix 44 near the ostium 42. The appendix 44 mat be stabilized by the magnetic union and may even be stretched or manipulated relative to the outside of the body by the magnetic retriever 34. Many structures in the body are not isolated or fixed in position in the body and may readily move inside a space such as the abdominal cavity. The ability to manipulate and hold steady a body cavity or organ such as the appendix may be helpful to assist in external ligation or visualization with an endoscope.

Another embodiment of the invention is shown in FIG. 3. A magnetic retriever 50 is shown having an elongated tubular body 52 with proximal 54 and distal ends 56. An expandable sheath 58 is connected to the distal end 56 of the body 52 and a retractable shaft 60 is positioned longitudinally inside the body and sheath. The shaft 60 is adapted to slide inside the inner lumen of the body 52 when activated by a handle 62. A magnetic coupler 64 is connected to the distal end of the shaft and configured to magnetically couple with a magnet such as magnet 31 across a tissue wall as previously described. The magnetic retriever 50 may be rigid to facilitate insertion through the skin and into the abdominal cavity similar to a trocar. Alternately the magnetic retriever may be flexible and steerable to facilitate placement through a natural body orifice. As the handle is retracted in a proximal direction the shaft and magnetic coupler are withdrawn into the expandable sheath 58 and body 52. Likewise a tissue, cavity or organ that is sandwiched between the magnetic coupling of the magnetic coupler 50 and the magnet 46 will be drawn inside the expandable sheath as well. The expandable sheath is designed to enlarge and expand as needed to envelop the removed organ as it is proximally pulled by the retriever.

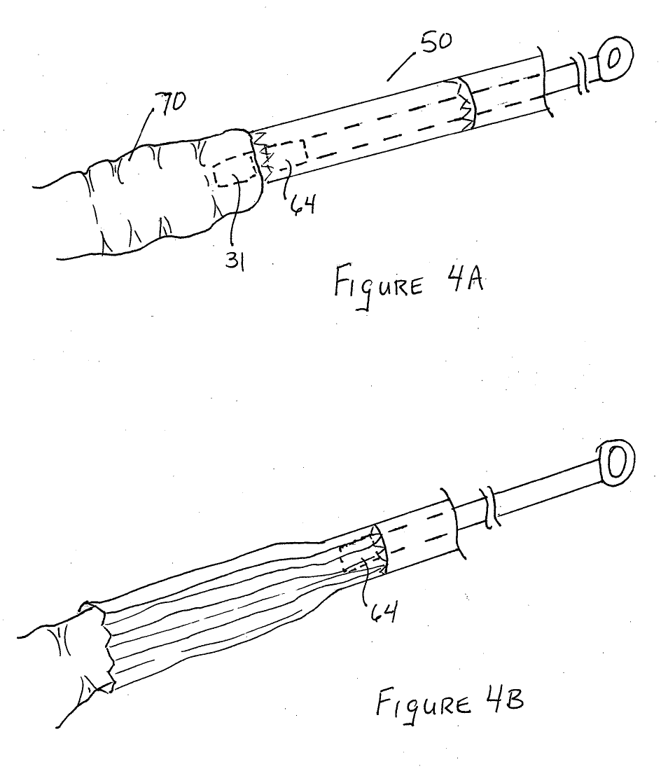

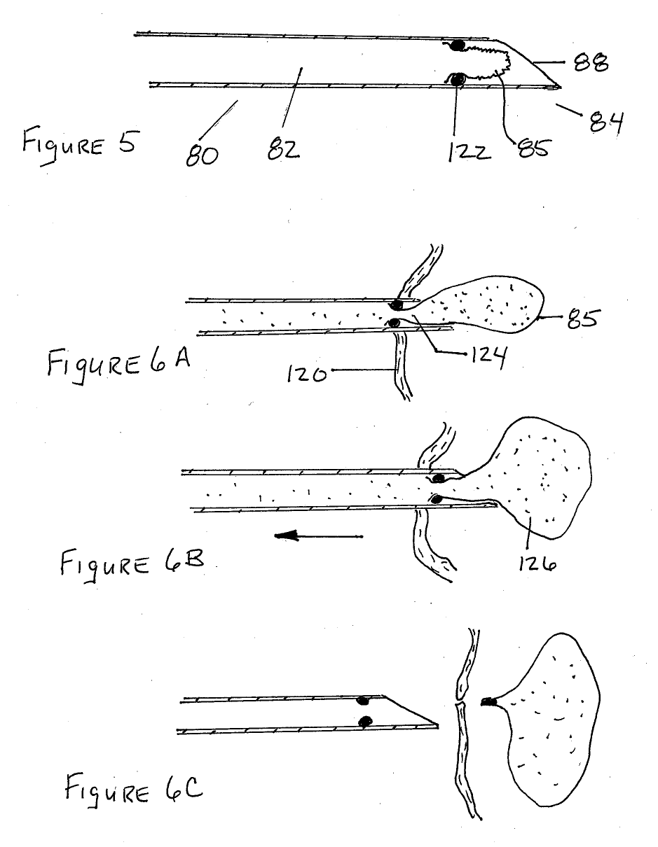

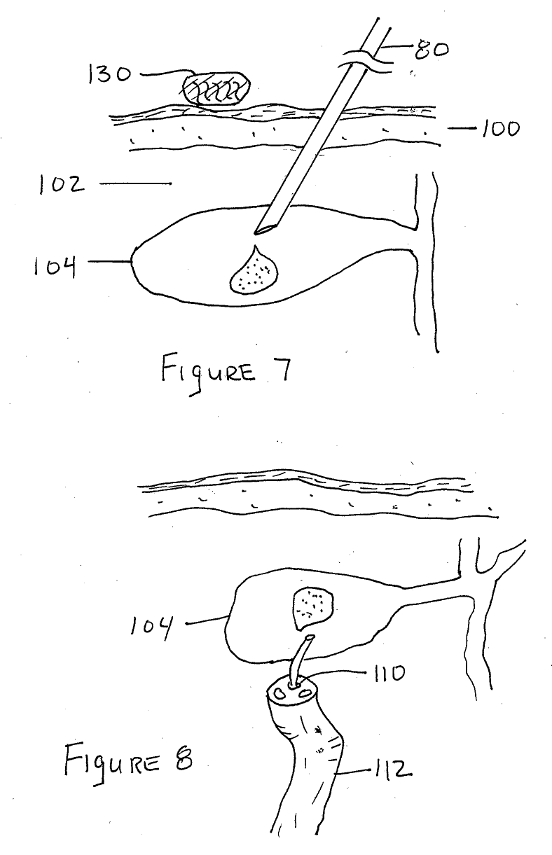

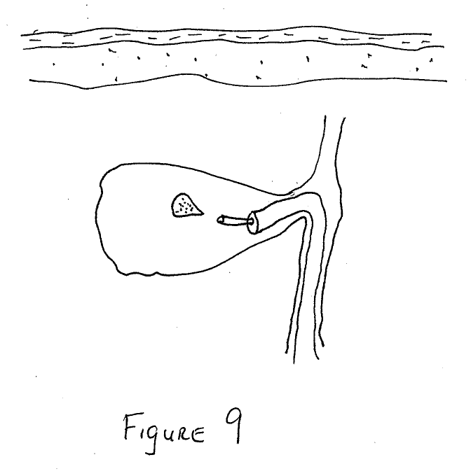

In one aspect of a method to remove a body cavity, tissue or organ, a magnetic coupler is shown in FIG. 4A, with a magnetic coupler 50 shown in close proximity to an organ 70 in the body. As the magnetic retriever 50 is brought closer to the organ 70 that has a magnet 31 positioned inside, the magnet 31 and the magnetic coupler 64 are attracted due to a magnetic force present between them and a magnetic coupling is made. At this point, the retriever 50 and the implanted magnet 31 are substantially joined together across the tissue wall. Obviously the force of attraction between the two elements is dependent on the strength of the magnets used and it is anticipated that various magnet strengths and types of magnets could be used. After the coupling has been accomplished, the organ or tissue may be manipulated as needed to position it in a satisfactory location for viewing with an endoscope, to move the organ out of the way so that other structures can be viewed or to facilitate ligation and resection of the organ or tissue. Additionally with the coupling accomplished, the handle 62 can be moved in a proximal direction relative to the elongated body to pull the magnetic coupler, tissue wall and the magnet inside the sheath. In this configuration the tissue is partially or wholly enveloped inside the sheath. If the tissue or organ has been resected from its attachment point to the body, then the complete withdrawal of the magnetic retriever from the body will also extract the tissue from the body. In one aspect of the invention the tissue or organ is ligated and resected prior to removal through the skin. Additionally in another aspect of the invention the tissue or organ may be pulled through the skin while still attached to the body and then the ligation and resection made outside the body where there is good visibility and access. By way of example, the appendix may be extracted using the retriever 50 and pulled outside the skin and then ligated and resected outside the body. The sheath may facilitate the removal of the tissue from the body by minimizing the size and the bulk of the tissue and by forming a smooth transition so that the tissue can be removed without hanging up or catching on other body structures. It is anticipated that the removal process may be facilitated by first debulking the tissue by the application of chemicals, suction or heat, cessation of blood supply or physical tissue removal. Using the appendix as an example, the appendix has a blood supply that must be managed and blocked before removal of the appendix using the techniques described. It might be advantageous to be able to locate and clamp off the blood vessels feeding the appendix through the intestinal wall transmurally or percutaneously.

Another embodiment of the invention that may be used to deliver a soft gel or fluid magnet, see FIG. 5, where a delivery device 80 is shown having an inner lumen 82 and a distal region 84. An inflatable balloon 85 is detachably coupled to the distal region 84 of the inner lumen 82 of the delivery device 80. The balloon is shown collapsed at least partially inside the delivery device 80. The delivery device distal region 84 may have a sharpened end 88 that may be used to introduce the delivery device 80 through the skin or other tissue and into the targeted tissue, organ or cavity. By way of example, the delivery catheter 80 may be introduced into a targeted organ such as the gall bladder. This may be accomplished by inserting the delivery catheter 80 through the layers of skin 100, into the abdominal cavity 102 and directly into a gall bladder 104 under fluoroscopic guidance as shown in FIG. 7. Alternatively the catheter 80 may be introduced through the use of endoscopy where the catheter is sized to be introduced into the working lumen 110 of an endoscope 112 as shown in FIG. 8. The endoscope 112 may be directed to the outer wall of the gall bladder 104 through a natural body orifice and then the catheter 80 may be extended through the wall of the gall bladder 104. In another embodiment of the invention, the catheter 80 may be introduced through the use of endoscopy where the catheter is sized to be introduced into the working lumen 110 of an endoscope 112 as shown in FIG. 9. The endoscope 112 may be directed through body vessels or ducts using a natural body orifice such as the mouth or rectum and then positioned into an adjacent duct or into a body cavity such as the gall bladder as shown. The catheter 80 may then be extended into the inner space of the gall bladder 104. Again, the gall bladder is provided for illustration only and it is anticipated that the catheter 80 could be introduced into any organ, tissue, cavity or hollow space that may require intervention.

Once the delivery catheter 80 is introduced inside the targeted organ, the deployment of the magnet can commence. FIG. 6A illustrates a delivery catheter 80 positioned across a wall of an organ 120. An inflatable balloon 85 is shown extruding from the distal region 84 of the delivery catheter. A closure element 122 positioned along the inner lumen 82 of the delivery catheter 80 is shown. This closure element may be used to seal the neck 124 of the balloon 85 once the balloon has been inflated to the desired size. The closure element 122 may also cut the balloon as it seals. The closure element 122 may be a band, suture, cinching device, clip, clamp, heat seal that fuses the sides of the neck 124 together or other commonly used element to seal balloons. The closure element may be aligned along the side walls of the inner lumen 82 or positioned in a recessed portion of the inner lumen walls. The closure device may be manually deployed by pulling a pull wire or activating a heating element or may be automatically deployed as the delivery catheter is removed from the gall bladder. Alternatively, as the balloon reaches a certain size, volume or extruded length outside the inner lumen 82 it may be deployed.

The balloon as described is intended to be used as part of a magnetic retrieval system and therefore needs to demonstrate magnetic properties to effectively couple with the opposing magnetic device. The balloon 85 may be inflated with a magnetic gel or fluid, either viscous or non viscous, 126 shown in FIG. 6B that is introduced into the inner lumen 82 at the proximal region of the delivery catheter 80 and flows into the neck 124 of the inflatable balloon near the closure element. The magnetic gel or fluid 126 may be a suspension of magnetic particles in a gel or fluid like material so that the gel or fluid suspension generates a magnetic field. Alternatively the magnetic gel or fluid 126 may be a suspension of ferrous particles that can be attracted by an opposing magnetic field. The density of the magnetic charge in these embodiments can be varied by increasing the density of the suspension in the gel or fluid. A gel or fluid is described because a gel suspension may provide a uniformity of dispersion of the magnetic particles. The gel described should be injectable with the ability to flow through the catheter to the inflatable balloon. Alternatively, the magnet 126 may be formed from a collection of magnetic or ferrous spheres that are introduced into the inflatable balloon 85 in a liquid carrier or this type of magnet could also be introduced into an organ or tissue without a balloon as stand alone spheres. These spheres may assume a collective compacted shape when in the presence of an external magnet that directs a magnetic field in their direction. However in the absence of such a magnetic field, the spheres may disperse inside the balloon or the organ. Because a liquid carrier may have potentially a lower viscosity than a gel, this may facilitate the removal of the organ from the body because the organ or tissue may more fully collapse and permit removal through a smaller incision in the skin.

In one embodiment, once the balloon is fully inflated, the delivery catheter is retracted in the direction of the arrow in FIG. 6B and pulled from the interior of the gallbladder. When this occurs, the closure element 122 seals the neck 124 of the balloon and the balloon is released into the gall bladder as shown in FIG. 6C. The size of the balloon may be controlled by including radiopaque particles in the gel suspension so that the balloon size can be seen and monitored using fluoroscopy.

In another embodiment of the magnet, a magnetic gel may be used but the gel is adapted for direct injection into the inner cavity or into the tissue walls using a needle and syringe or a trocar-like introducer. This embodiment does not require a delivery catheter or inflatable balloon so the devices required may be less expensive and the procedure might be simpler. In another embodiment the gel may be introduced as a liquid and then solidify over a period of time. This type of gel would be injectable yet would be less likely to migrate from the original injection site over time.

In another embodiment of the invention, a magnetic balloon 85 is placed into an organ or body cavity and may be coupled to an external magnet 130 positioned on the skin 100 as shown in FIG. 7. The external magnet 130 may be a very powerful magnet compared to endoscopic magnets previously described. This increased power may be possible because the external magnet can be quite large and the size is not limited by the size limitations of a magnet placed inside the body. As such it is conceivable that a strong magnetic field could be generated by this external magnet 130 and provide a strong coupling force for the magnetic balloon 85.

Certain objects and advantages of the invention are described herein. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

All of these embodiments are intended to be within the scope of the present invention herein disclosed. However, despite the foregoing discussion of certain embodiments, only the appended claims (and not the present summary) are intended to define the invention. The summarized embodiment, and other embodiments of the present invention, will become readily apparent to those skilled in the art from the following detailed description of he preferred embodiments having reference to the attached figures, the invention not being limited to any particular preferred embodiment(s) disclosed.

What is claimed is:

1. A magnetic retrieval device to stabilize or manipulate a body cavity comprising:
a magnet configured for delivery to the inside of the body cavity,
a delivery device to deliver the magnet to the body cavity,
a magnetic retriever configured to magnetically couple with the magnet through a wall of said body cavity,
and the coupled magnetic retriever and magnet adapted to control the position of the body cavity relative to the outside of the body.

2. The magnet of claim 1 further comprising a balloon, said balloon having a first deflated condition inside the delivery device and a second inflated condition whereby the balloon extrudes from the inner lumen of the delivery device,
a magnetic fluid adapted to inflate the balloon,
and a balloon closure element to seal the balloon.

3. The magnetic fluid of claim 2 wherein the fluid is a gel.

4. The magnetic fluid of claim 2 wherein the fluid is a ferromagnetic material dispersed within a fluid medium, said ferromagnetic material adapted to migrate within the fluid medium to form a unitary mass in the presence of a magnetic field.

5. The balloon of claim 4 wherein the balloon is detachably coupled to a distal region of an inner lumen of the delivery device.

6. The delivery device of claim 1 wherein the delivery device is sized for delivery through the working channel of an endoscope and configured to pierce the wall of the body cavity.

7. The magnetic retriever of claim 2 wherein the magnetic retriever is a polar opposite magnet positioned on the skin.

8. The magnetic retriever of claim 2 wherein the magnetic retriever is adapted to be placed through a natural body orifice to the body cavity.

9. The magnet of claim 1 further comprising a magnetic fluid having a first condition whereby the fluid is injectable into the body cavity, and the fluid changeable into a second condition whereby the fluid becomes a solid.

10. The delivery device of claim 1 further comprising a guidewire having a distal end, the magnet and the guidewire being detachably coupled at said distal end such that the magnet can be decoupled from the guidewire thus releasing the magnet so that the guidewire may be withdrawn.

11. The magnetic retriever of claim 1 further comprising an elongated tubular body having proximal and distal ends,
an expandable sheath attached to the distal end of the tubular body,
a retractable shaft having distal and proximal ends positioned longitudinally inside the body and sheath,
a magnet coupler connected to the distal end of the shaft and configured to magnetically couple with the magnet,
the shaft configured to retract proximally inside the sheath and tubular body once coupled to the magnet to draw the magnet and the body cavity into the expandable sheath.

12. A method of stabilizing or manipulating a body cavity comprising:
positioning a delivery device, having a magnet, inside the body cavity,
delivering said magnet to the cavity,
magnetically coupling to the magnet with a magnetic retriever positioned outside the body cavity and manipulating the body cavity by manipulating said coupling.

13. The method of claim 12 wherein delivering the magnet includes inflating a balloon positioned at a distal end portion of the delivery device with a magnetic fluid, sealing a neck of the balloon with a closure element and releasing the balloon into the body cavity.

14. The method of claim 13 wherein the magnetic fluid is a ferromagnetic material dispersed within a fluid medium, said ferromagnetic material adapted to migrate within the fluid medium to form a unitary mass in the presence of a magnetic field.

15. The method of claim 12 wherein delivering the magnet includes releasing a ferromagnetic element detachably connected to a distal end portion of the delivery device into the body cavity, and withdrawing the delivery device.

16. The method of claim 15 wherein said delivery device is a guidewire.

17. The method of claim 12 further comprising manipulating the body cavity to facilitate resection of the body cavity.

18. The method for manipulating the body cavity of claim 17 wherein said resection is accomplished outside the body.

19. A method of resecting a body cavity or organ comprising:
positioning a delivery device, having a magnet, inside the body cavity or organ, delivering said magnet to the body cavity or organ,
positioning a magnetic retriever outside the body cavity or organ, said retriever comprising an elongated tubular body having proximal and distal ends, with an expandable sheath attached to the distal end of the tubular body, a retractable shaft having distal and proximal ends positioned longitudinally inside the body and sheath and a magnet coupler connected to the distal end of the shaft,
magnetically coupling to the magnet with the magnetic coupler,
drawing the body cavity or organ into the expandable sheath by moving the shaft proximally inside the elongated tubular body,
and resecting the body cavity or organ from the body.

20. The method of claim 19 further comprising ligating a base of the body cavity or organ prior to the resection.

21. The method of claim 20 wherein the body cavity is an appendix or gallbladder.

22. The method of claim 21 wherein the expandable sheath encases the appendix or gall bladder and removing the magnetic retriever includes pulling the device through the skin.

23. A system for removing tissue from the body comprising:
a magnet configured for delivery inside the tissue,
a delivery device to deliver the magnet into the tissue,
a magnetic retriever configured to magnetically couple with the magnet across a wall of said tissue,
said magnetic coupling adapted to manipulate the tissue to facilitate resection of the tissue.

24. The system of claim 23 further comprising a tissue ligating element to ligate the tissue before resecting the tissue and a tissue resecting element to resect the tissue from the body.

25. The magnet of claim 23 further comprising an inflatable balloon, an inflation neck of said balloon detachably coupled to a distal region of an inner lumen of the delivery device, said balloon having a first deflated condition inside the delivery device and a second inflated condition whereby the balloon extrudes from the inner lumen of the delivery device,
a magnetic gel adapted to inflate and fill the balloon,
and a balloon closure element to seal the inflation neck of the balloon and detach said balloon from the delivery catheter.
